(19) United States
(12) Patent Application Publication
(10) Pub. No.: US 2024/0198034 A1
(43) Pub. Date: Jun. 20, 2024

(54) PLATFORM PROVIDING SYSTEM FOR VEHICLE CONTENT SERVICE AND PLATFORM PROVIDING METHOD THEREFOR

(71) Applicants: HYUNDAI MOTOR COMPANY, Seoul (KR); KIA CORPORATION

(72) Inventors: Ki Chang Kim, Suwon-si (KR); Dong Chul Park, Anyang-si (KR); Eun Soo Jo, Hwaseong-si (KR); Sang Jin Hong, Hwaseong-si (KR)

(73) Assignees: HYUNDAI MOTOR COMPANY, Seoul (KR); KIA CORPORATION, Seoul (KR)

(21) Appl. No.: 18/228,840

(22) Filed: Aug. 1, 2023

(30) Foreign Application Priority Data
Dec. 16, 2022 (KR) 10-2022-0177522

(51) Int. Cl. A61M 21/02; G06Q 30/0601; G06Q 50/00

(52) U.S. Cl. CPC A61M 21/02; G06Q 30/0621; G06Q 50/01; A61M 2021/0027; A61M 2205/3375; A61M 2230/06; A61M 2230/10

(57) ABSTRACT

A platform providing system and a platform providing method are disclosed for a vehicle content service to provide a virtual sound design platform using a non-fungible token (NFT)-based blockchain technology. The platform providing system includes a service platform server that constructs a service platform in a metaverse-based virtual environment and includes a blockchain platform server that supports the service platform. The service platform server generates a marketplace by means of a smart contract-based commerce platform in conjunction with the blockchain platform server, generates a smart healing function based on a user experience analysis of a virtual vehicle in the service platform, and generates a sound design platform supporting to design a virtual sound in the service platform and transact the virtual sound in the marketplace.

### CROSS-REFERENCE TO RELATED APPLICATION

This application claims the benefit of and priority to Korean Patent Application No. 10-2022-0177522, filed in the Korean Intellectual Property Office on Dec. 16, 2022, the entire contents of which are incorporated herein by reference.

### TECHNICAL FIELD

The present disclosure relates to a platform providing system for a vehicle content service to provide a virtual sound design platform using a non-fungible token (NFT)-based blockchain technology and a platform providing method therefor.

### BACKGROUND

A non-fungible token (NFT) refers to a virtual token which proves ownership of digital assets (e.g., content such as drawings, photos, sound sources, images, or videos) using a blockchain technology. In other words, the NFT is a digital token to which the blockchain technology is applied, which contains a unique recognition value.

The statements in this section merely provide background information related to the present disclosure and may not constitute prior art.

### SUMMARY

An aspect of the present disclosure provides a platform providing system for a vehicle content service to provide a vehicle content service platform for designing and possessing or transacting a virtual sound in a virtual environment using an NFT-based blockchain technology and a platform providing method therefor.

The technical problems to be solved by the present disclosure should be clearly understood: the following description by those having ordinary skill in the art to which the present disclosure pertains.

According to an aspect of the present disclosure, a platform providing system for a vehicle content service may include a service platform server that constructs a service platform in a metaverse-based virtual environment. The platform providing system may also include a blockchain platform server that supports the service platform. The service platform server may generate a marketplace by means of a smart contract-based commerce platform in conjunction with the blockchain platform server. The service platform server may also generate a smart healing function based on a user experience analysis of a virtual vehicle in the service platform. The service platform server may also generate a sound design platform supporting to design a virtual sound in the service platform and transact the virtual sound in the marketplace.

The service platform server may generate a transaction model supporting to perform a content transaction in conjunction with the blockchain platform server.

The transaction model may support a content transaction using a non-fungible token (NFT)-based blockchain technology.

The service platform server may analyze a driving pattern of a user based on driving data of the virtual vehicle and may establish an emotion modeling concept depending on the driving pattern.

The service platform server may generate determination logic for determining cyber sickness based on a biometric signal of the user using a biometric signal measurement device. The service platform server may also generate cyber sickness mitigation control logic in response to that an occurrence of the cyber sickness.

The biometric signal measurement device may include an electroencephalogram (EEG) measurement device that measures EEG in a dry manner and may include a haptic controller that measures a heart rate.

The EEG measurement device may include a microphone that measures a breathing sound.

The service platform server may determine a user state by analyzing a biometric signal of a user. The service platform server may also derive an emotional concept based on the user state. The service platform server may also generate sound masking emotion content using white noise or pink noise based on the emotional concept.

The service platform server may measure a sound level using a sensor installed on a front upper end in the interior of a vehicle. The service platform server may also play a masking sound based on the measured sound level. The service platform server may also output the played masking sound through a front mid-range speaker of a vehicle.

The service platform server may measure a sound level using a sensor installed on an ear position of the user. The service platform server may also play a masking sound based on the measured sound level. The service platform server may also output the played masking sound through a speaker installed on an ear position of the user.

According to another aspect of the present disclosure, a platform providing method for a vehicle content service may include constructing, by a service platform server, a service platform in a metaverse-based virtual environment. The platform providing method may also include generating, by the service platform server, a marketplace by means of a smart contract-based commerce platform in conjunction with a blockchain platform server. The platform providing method may also include generating, by the service platform server, a smart healing function based on a user experience analysis of a virtual vehicle in the service platform. The platform providing method may also include generating, by the service platform server, a sound design platform supporting to design a virtual sound in the service platform and transact the virtual sound in the marketplace.

The constructing of the vehicle content platform may include generating a transaction model supporting to perform a content transaction in conjunction with the blockchain platform server.

The transaction model may support a content transaction using a non-fungible token (NFT)-based blockchain technology.

The generating of the smart healing function may include analyzing, by the service platform server, a driving pattern of a user based on driving data of the virtual vehicle and establishing, by the service platform server, an emotion modeling concept depending on the driving pattern.

The generating of the smart healing function may include generating determination logic for determining cyber sickness based on a biometric signal of the user and generating cyber sickness mitigation control logic in response to that an occurrence of the cyber sickness.

The generating of the sound design platform may include determining a user state by analyzing a biometric signal of a user, deriving an emotional concept based on the user state, and generating sound masking emotion content using white noise or pink noise based on the emotional concept.

### BRIEF DESCRIPTION OF THE DRAWINGS

The above and other objects, features, and advantages of the present disclosure should be more apparent from the following detailed description taken in conjunction with the accompanying drawings:

FIG. 1 is a block diagram illustrating a configuration of a platform providing system for a vehicle content service according to embodiments of the present disclosure;

FIG. 2 is a drawing illustrating an emotional content development process according to embodiments of the present disclosure;

FIG. 3 is a drawing illustrating a process for developing a smart healing function by means of user experience analysis-based control according to embodiments of the present disclosure;

FIG. 4 is a drawing illustrating an example of a sound design platform according to embodiments of the present disclosure;

FIG. 5 is a drawing illustrating another example of a sound design platform according to embodiments of the present disclosure;

FIG. 6 is a drawing illustrating a biometric signal measurement device according to embodiments of the present disclosure;

FIG. 7 is a drawing illustrating an emotional content development process based on a natural sound-based stress relief and meditation concept according to embodiments of the present disclosure;

FIG. 8 is a graph illustrating a sound level according to frequencies of white noise and pink noise associated with the present disclosure;

FIG. 9 is a graph illustrating a sound level according to sound masking according to an embodiment of the present disclosure; and

FIG. 10 is a flowchart illustrating a sound masking method according to embodiments of the present disclosure.

### DETAILED DESCRIPTION

Hereinafter, some embodiments of the present disclosure are described in detail with reference to the drawings. In the drawings, the same reference numerals are used throughout to designate the same or equivalent elements. In addition, a detailed description of well-known features or functions has been omitted in order not to unnecessarily obscure the gist of the present disclosure.

In describing the components of the embodiment according to the present disclosure, terms such as first, second, “A”, “B”, (a), (b), and the like may be used. These terms are only used to distinguish one element from another element and do not limit the corresponding elements irrespective of the order or priority of the corresponding elements. Furthermore, unless otherwise defined, all terms including technical and scientific terms used herein should be interpreted as customary in the art to which the present disclosure belongs. Such terms as those defined in a generally used dictionary should be interpreted as having meanings equal to the contextual meanings in the relevant field of art and should not be interpreted as having ideal or excessively formal meanings unless clearly defined as having such in the present application.

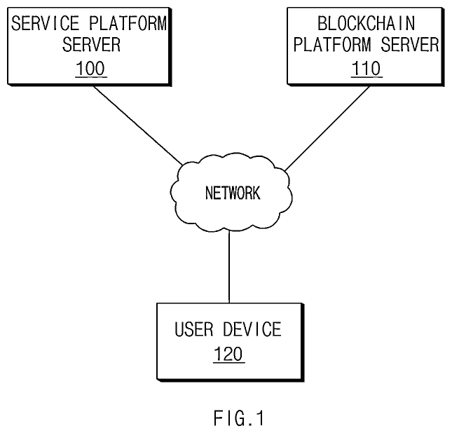

FIG. 1 is a block diagram illustrating a configuration of a platform providing system for a vehicle content service according to embodiments of the present disclosure.

Referring to FIG. 1, the platform providing system for the vehicle content service may include a service platform server 100, a blockchain platform server 110, and a user device 120, which are connected with one another over a wired and/or wireless network. Each of the service platform server 100, the blockchain platform server 110, and the user device 120 may include at least one processor, a memory, a communication circuit, and the like. The at least one processor may include at least one of processing devices, such as an application specific integrated circuit (ASIC), a digital signal processor (DSP), a programmable logic device (PLD), a field programmable gate array (FPGA), a central processing unit (CPU), a microcontroller, or a microprocessor. The memory may be a non-transitory storage medium, which stores instructions executed by the at least one processor. The memory may be implemented as at least one of storage media (or recording media), such as a flash memory, a hard disk, a solid state disk (SSD), a secure digital (SD) card, a random access memory (RAM), a static RAM (SRAM), a read only memory (ROM), a programmable ROM (PROM), an electrically erasable and programmable ROM (EEPROM), an erasable and programmable ROM (EPROM), a removable disk, or web storage. The communication circuit may support communication among the service platform server 100, the blockchain platform server 110, and the user device 120. The communication wireless may include a wireless communication circuit (e.g., a long term evolution (LTE) communication circuit, a short range communication circuit, a Wi-Fi communication circuit, or the like), a wired communication circuit (e.g., a local area network (LAN) communication circuit, a wide area network (WAN) communication circuit, an Ethernet communication circuit, a power line communication circuit, or the like).

The service platform server 100 may implement a service platform, for example, a vehicle lab platform, a vehicle emotional design platform (or a virtual sound design platform), and/or the like in a metaverse-based virtual environment. The vehicle lab platform may support content activities, such as a vehicle disassembly and assembly academy, driving education in a virtual environment, a racing car, a driver test, and/or a driving sound design in a virtual environment. The vehicle emotional design platform may support to create (or design) and share an electronic sound, a warning sound, and/or a virtual sound by developing emotional content with smart healing concept and designing a virtual environment sound in conjunction with a metaverse and a blockchain.

The service platform server 100 may transmit information, such as member information of a user (i.e., an individual member) who subscribes to the vehicle lab platform implemented in the virtual environment, a creative sound (or auctionable content), and/or a point (or a virtual cache capable of being replaced with a token), to the blockchain platform server 110. The member information is information for identifying a user, and the creative sound is a virtual sound designed by the user.

The service platform server 100 may assign one virtual vehicle to each of users in the virtual environment (i.e., the vehicle lab platform) to provide a token (or a point) depending on content activity and thus may support to level up a vehicle design, power, and/or function. The content activity may be a vehicle disassembly and assembly academy, driving education in a virtual environment, a racing car, a driver test, a driving sound design in a virtual environment, and/or the like.

The service platform server 100 may provide a sound design tool through the virtual sound design platform. The user may access the virtual sound design platform through the user device 120 to use the sound design tool.

The service platform server 100 may provide a racing car-based play culture on the vehicle lab platform, may enable an ultra-realistic sound design to provide an ultra-realistic sound, and may develop personalization technology.

The blockchain platform server 110 may support the vehicle lab platform and may be compatible with the service platform server 100. The blockchain platform server 110 may receive the information, such as the member information of the user, the creative sound, and/or the point from the service platform server 100. The blockchain platform server 110 may encrypt the pieces of received information using a blockchain technology.

The blockchain platform server 110 may maintain transparent transaction details using a distributed ledger technology (DLT). The blockchain platform server 110 may include a token generator, a data management device, a metamask cryptocurrency transaction device to collect, sell, or share creative content (e.g., a creative sound, an electronic sound, a warning sound, a virtual sound, or the like) based on a virtual world token (e.g., an NFT). The token generator may create (or generate) an NFT linked to content (e.g., a creative sound or the like) requested by the user device 120. The token generator may generate an NFT based on information, such as the member information of the user, the creative sound, and/or a point, which are transmitted from the service platform server 100. The data management device may manage an asset to the user. The metamask cryptocurrency transaction device may store, transact, and manage cryptocurrency using a metamask.

The blockchain platform server 110 may transmit data, such as a metamask (which is a bankbook concept and is used for a cryptocurrency transaction), auction data information (based on the DLT), and/or a token (i.e., cryptocurrency capable of being replaced with a point), to the service platform server 100.

The blockchain platform server 110 may support to be linked with the service platform server 100 to perform a content transaction in an NFT-based marketplace in the virtual environment.

The user device 120 may be an electronic device used by a user (or a virtual vehicle driver) who subscribes to the vehicle lab platform constructed by the service platform server 100. The user device 120 may be an audio video navigation (AVN) device, a smartphone, a tablet, a laptop, a computer, or the like. Only one user device 120 is illustrated in the drawing, but not limited thereto. The user device 120 may be composed of two or more user devices 120.

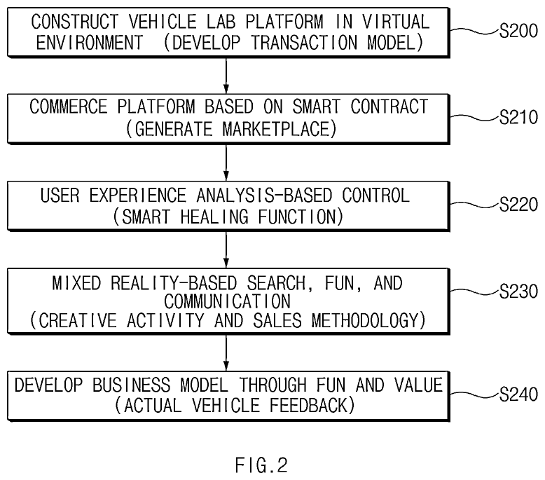

FIG. 2 is a drawing illustrating an emotional content development process according to embodiments of the present disclosure.

In operation S200, a service platform server 100 of FIG. 1 may construct a vehicle lab platform in a metaverse-based virtual environment. The vehicle lab platform may support content activities, such as a vehicle disassembly and assembly academy, driving education in a virtual environment, a racing car, and a driver test. The service platform server 100 may generate a transaction model (or a business model), which supports to perform a content transaction in conjunction with a blockchain platform server 110 of FIG. 1.

In operation S210, the service platform server 100 may interwork with the blockchain platform server 110, which supports the vehicle lab platform, and may generate a marketplace by means of a smart contract-based commerce platform. Herein, the smart contract refers to concluding and fulfilling a contract for a content transaction based on a blockchain. A driving sound, a warning sound, an electronic sound, and/or the like generated by means of a sound design may be transacted in the marketplace. The commerce platform may provide an information community by means of a blockchain-based marketplace.

In operation S220, the service platform server 100 may develop a smart healing function using user experience analysis-based control. The smart healing function may provide a driver emotion modeling-based sound. When generating the smart healing function, the service platform 100 server may generate determination logic for determining cyber sickness based on a biometric signal of the user and cyber sickness mitigation control logic.

In operation S230, the service platform server 100 may provide a creative activity and sales methodology through mixed reality-based search, fun, and communication.

In operation S240, the service platform server 100 may develop a business model through fun and value (or a business model through actual vehicle feedback). The developed business model may be a virtual sound design model for vehicle, which may provide a virtual sound design program to enable a design creation activity for a vehicle electronic sound, a warning sound, and a virtual sound and may support to enable sales and personalization sharing. In other words, the service platform server 100 may support to possess or sell a creative sound by means of the developed business model and load the creative sound as a personalized sound into a real-world vehicle using a communication technology.

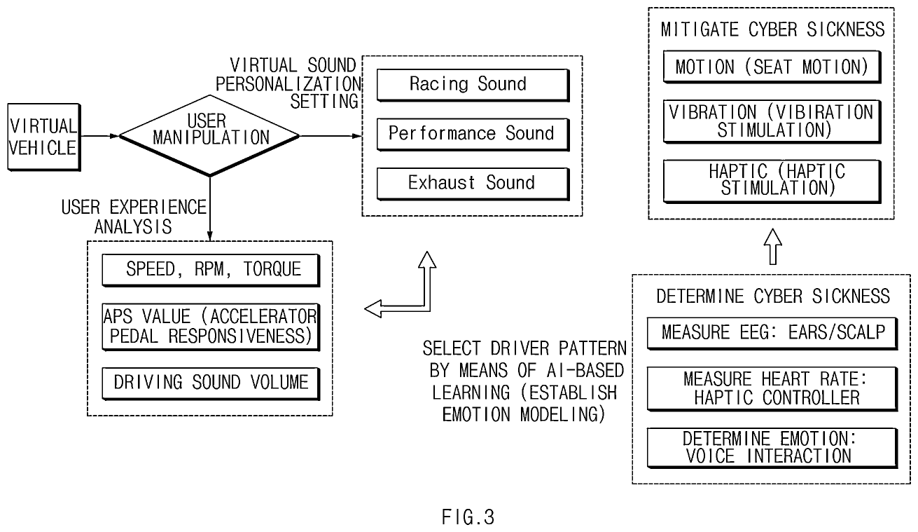

FIG. 3 is a drawing illustrating a process for developing a smart healing function by means of user experience analysis-based control according to embodiments of the present disclosure.

A service platform server 100 of FIG. 1 may detect virtual vehicle manipulation of a user (or a driver or an individual member) in a metaverse-based virtual environment. The service platform server 100 may receive data according to user manipulation (or a user input) from a user device 120 of FIG. 1, which is matched with a virtual vehicle. The service platform server 100 may select a user experience analysis or function a virtual sound personalization setting function depending on user manipulation.

When the user experience analysis function is selected, the service platform server 100 may analyze driving data according to virtual vehicle driving of a user, for example, a vehicle speed, an engine revolution per minute (RPM), engine torque, accelerator pedal responsiveness (e.g., an accelerator position sensor (APS) value), driving sound volume, and the like.

When the virtual sound personalization setting function is selected, the service platform server 100 may analyze a sound preference of the user for a high-performance vehicle concept, for example, a racing sound, a performance sound, or an exhaust sound.

The service platform server 100 may select a driving pattern of the user based on driving data. At this time, the service platform server 100 may select the driving pattern of the user by means of artificial intelligence (AI)-based learning. In other words, the service platform server 100 may select a vehicle speed, an RPM, torque, an APS value, a driving sound volume preference, and the like as parameters using a deep learning technology of an AI concept. The service platform server 100 may determine a driving pattern as a constant speed mode, an acceleration mode, or the like by means of an AI mode learned based on the selected parameter. Furthermore, the service platform server 100 may establish an emotional model by selecting the driving pattern of the user.

The service platform server 100 may proceed with a user experience analysis by user manipulation in a virtual vehicle and may establish user emotion modeling in three ways. A cultured sound type of user may prefer soft, low-mid, and high-quality sound, which is a harmony concept. An entertaining sound type of user may prefer a fun futuristic sound, which is a fun driving concept. An adaptable sound type of user may prefer a sound which changes according to the user's mood, which is a context awareness concept. The user may enjoy a racing sound, a performance sound, or an exhaust sound by recommending an AI-based personalization sound. The service platform server 100 may mount a creative sound on a vehicle personalization sound by means of a virtual and vehicle environment sound design program.

The service platform server 100 may determine and mitigate cyber sickness of the user. The service platform server 100 may determine whether sickness an cyber occurs using electroencephalogram (EEG) measurement device and may identify (or detect) a time point when the cyber sickness occurs, a cyber sickness duration, and the like. The EEG measurement device may measure EEG in a dry type on ears and/or a scalp (or forehead). Furthermore, the service platform server 100 may measure a heart rate by means of a haptic controller held in the user's hand and may determine emotion of the user using voice interaction-based audio and/or text data.

The service platform server 100 may provide an immersion technology using the five senses to mitigate cyber sickness and discover surreal emotion. The service platform server 100 may mitigate cyber sickness by means of seat motion, vibration stimulation, haptic stimulation, or the like.

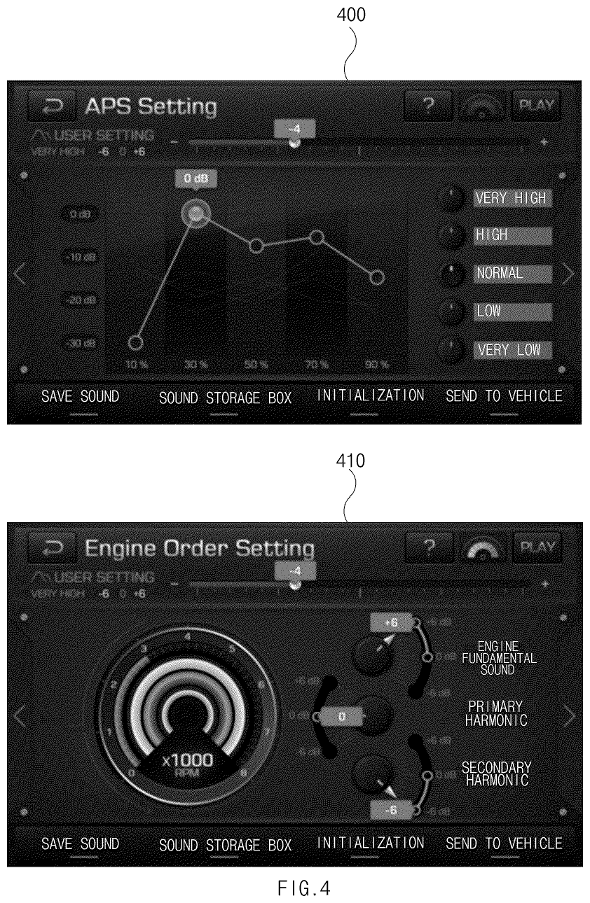

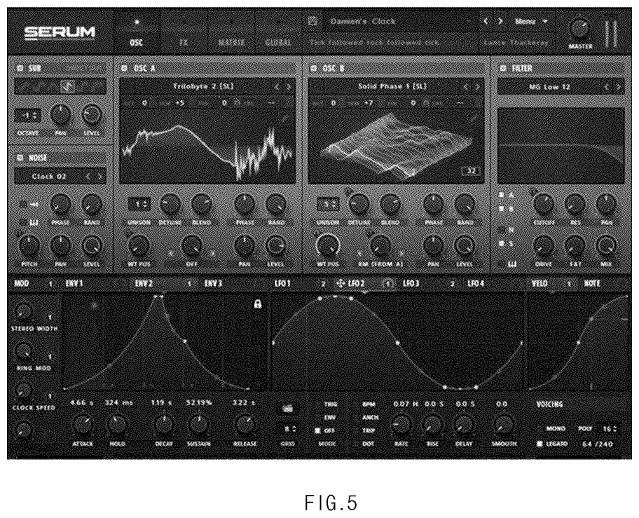

FIG. 4 is a drawing illustrating an example of a sound design platform according to embodiments of the present disclosure. FIG. 5 is a drawing illustrating another example of a sound design platform according to embodiments of the present disclosure.

As shown in FIG. 4, a service platform server 100 of FIG. 1 may provide a user device 120 of FIG. 1 with sound design platforms 400 and 410 for general user, for allowing a general user to simply design a sound. A user device 120 of FIG. 1 may output the APS setting platform 400, the engine order setting platform 410, and the like transmitted from the service platform server 100 on its screen. A user may adjust APS resistance in the APS setting platform 400 and may adjust engine sound volume according to an engine order in the engine order setting platform 410.

Furthermore, as shown in FIG. 5, the service platform server 100 may provide a sound design platform for expert. An expert may adjust a sound in more detail to create a virtual sound in the sound design platform for expert.

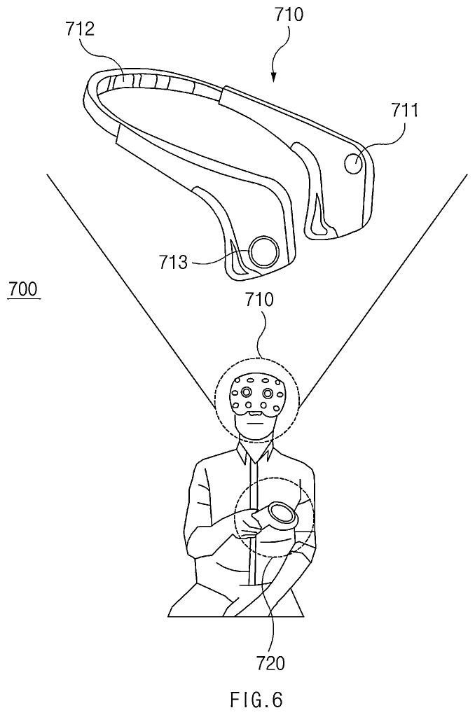

FIG. 6 is a drawing illustrating a biometric signal measurement device according to embodiments of the present disclosure.

Referring to FIG. 6, a biometric signal measurement device 700 may include an EEG measurement device 710 and a haptic controller 720.

The EEG measurement device 710 may be a wearable device capable of being worn on the head of a user, which may be manufactured in the form of a headband. The EEG measurement device 710 may measure EEG of the user using a dry EEG measurement scheme. The EEG measurement device 710 may measure EEG using a first sensor 711 in contact with an ear area and a second sensor 712 in contact with a scalp (e.g., a forehead).

The EEG measurement device 710 may further include a microphone 713 for measuring a breathing rate and/or a breathing sound of the user. The breathing rate may be used to measure a health state of the user. The breathing sound may be used to determine a stress degree of a driver or diagnose an emotional state depending on a pattern analysis of a sound source. The EEG measurement device 710 may determine a stress degree and/or an emotional state of the driver based on the loudness and roughness of the breathing sound.

When the palm of the user is in contact with a sensor (or an electrode) installed on a handle portion, the haptic controller 720 may measure a heart rate (or a maximum heart rate) of the user. The heart rate may also be measured on the wrist or ear. The heart rate may be used to determine a stress degree of the user or diagnose an emotional state of the user.

The biometric signal measurement device 700 may develop emotional content of a smart healing concept based on a user's body information analysis technology. In other words, the biometric signal measurement device 700 may develop an immersion enhancement technology for analyzing EEG, a heart rate, a breathing sound, and/or the like of the user, determining whether cyber sickness occurs based on the analyzed result, and preventing cyber sickness.

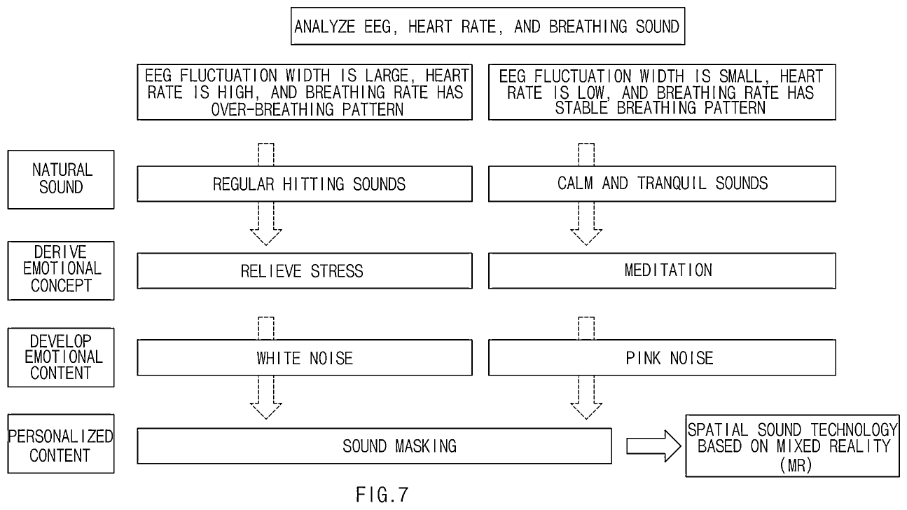

FIG. 7 is a drawing illustrating an emotional content development process based on a natural sound-based stress relief and meditation concept according to embodiments of the present disclosure.

A service platform server 100 of FIG. 1 may analyze a biometric signal such as EEG, a heart rate, a breathing rate, and/or a breathing sound of a user (or a driver), which are/is obtained by means of a biometric signal measurement device 700 of FIG. 6. The service platform server 100 may determine whether the user is in a stress situation or in a state in need of psychological stability depending on the analyzed result.

The service platform server 100 may classify a natural sound pattern based on the result of analyzing the biometric signal. For example, when an EEG fluctuation width is large, when the heart rate is high, and the breathing rate has an over-breathing pattern, the service platform server 100 may classify the natural sound pattern into regular hitting sounds (e.g., the sound of rain, the sound of waves, the sound of mountain temple bells, or the sound of birds). When the EEG fluctuation width is small, when the heart rate is low, and the breathing rate has a stable breathing pattern, the service platform server 100 may classify the natural sound pattern into calm and tranquil sounds (e.g., the sound of the forest, the sound of the stream, the sound of the wind, or the sound of grass insects). The service platform server 100 may analyze a correlation between biometric information and a natural sound and may derive (or determine) an emotional concept. When the natural sound pattern is classified into the regular hitting sounds by analyzing the correlation between the biometric information and the natural sound, the service platform server 100 may determine an emotional concept as “stress mitigation”. When the natural sound pattern is classified into the calm and tranquil sounds by analyzing the correlation between the biometric information and the natural sound, the service platform server 100 may determine an emotional concept as “meditation”.

The embodiment is not limited to the case where the user state is determined using the biometric information in the present embodiment. An embodiment may be implemented to determine a user state using a user face expression recognition technology and/or a user voice interaction technology. The service platform server 100 may generate an emotional care sound based on a natural sound using a sound design tool. The natural sound may include white noise and pink noise. The white noise is noise in an electronic device due to electron free motion and has a uniform spectrum for a frequency. The white noise may be associated with regular hitting sound (s), such as the sound of rain, the sound of waves, the sound of mountain temple bells, and/or the sound of birds. Because of having a non-constant sound band width and a wide spectrum characteristic, the white noise may relieve stress and may improve mental stability and concentration. The pink noise is noise, which has the same amount of energy every octave and is used as a sound source for test, such as sound characteristic measurement. The pink noise is associated with calm and tranquil sounds, such as the sound of the forest, the sound of the stream, the sound of the wind, and/or the sound of grass insects. The pink noise helps to sleep in conjunction with an autonomous sensory meridian response (ASMR), which is a sound configured to stimulate the brain and induce psychological stability and is highly related to meditation which gives psychological stability.

The service platform server 100 may recommend an AI-based personalized healing sound in a virtual environment and/or a vehicle environment. The service platform server 100 may provide an emotional care sound based on white noise or an emotional care sound based on pink noise depending on a user state. The service platform server 100 may recommend the emotional care sound as a healing sound and may block ambient noise, which is not desired by the user to listen, by means of sound masking.

The service platform server 100 may develop sound masking emotion content by means of white noise and pink noise. The sound masking may block unwanted ambient noise by neutralizing sounds that the user does not want to hear or disturbing sounds. The service platform server 100 may provide a spatial sound technology based on mixed reality (MR) using the sound masking.

When providing a user (e.g., a driver) with the emotional care sound based on the white noise or the emotional care sound based on the pink noise, the service platform server 100 may apply a sound masking technology to a vehicle such that a passenger (e.g., a passenger who sits on a passenger seat or a rear seat) except for the user (e.g., the driver) does not feel the sound as annoying noise.

As an embodiment, the service platform server 100 may measure a sound level (or a noise level) using a sensor installed on a front upper end in the interior of the vehicle with regard to an operation mode (e.g., a normal mode and a sleep mode) of a seat and a situation where users ride in all of front seats and rear seats. The service platform server 100 may play a masking sound source based on the measured sound level. The service platform server 100 may output the played masking sound to the outside through a front mid-range speaker of the vehicle.

As another embodiment, the service platform server 100 may sense a sound (or a sound level or a noise level) using a sensor installed (or located) at a user's ear position. The service platform server 100 may play a masking sound source based on the sensed sound. The service platform server 100 may output the played masking sound through a speaker installed (or located) at the user's ear position.

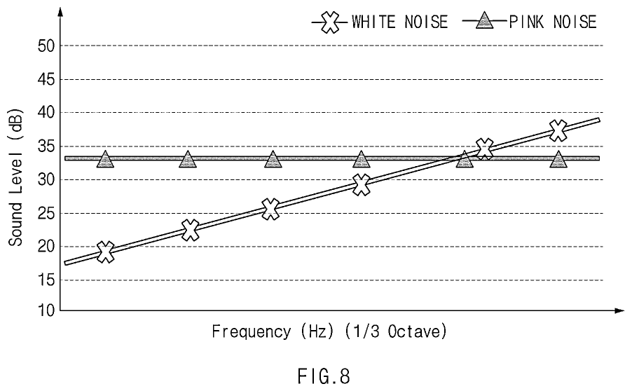

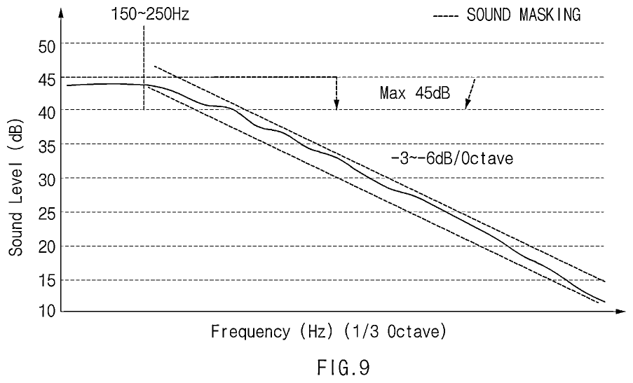

FIG. 8 is a graph illustrating a sound level according to frequencies of white noise and pink noise associated with the present disclosure. FIG. 9 is a graph illustrating a sound level according to sound masking according to an embodiment of the present disclosure.

Referring to FIG. 8, white noise has the same energy for each frequency in a narrow band, and pink noise has the same energy in an octave band.

Referring to FIG. 9, it may be identified that the effect of evaluated and verified sound masking is excellent through a user experience (UX). The sound masking may set a maximum level of a sound to 45 dB for hearing organ care and may suppress unwanted ambient noise at 3-6 db/octave in a region of 150 Hz to 250 Hz to maximize a masking effect. Such sound masking is a technology for outputting another sound not to recognize a specific sound (e.g., a human voice), which may be applied to an office environment or the like as well as a vehicle environment.

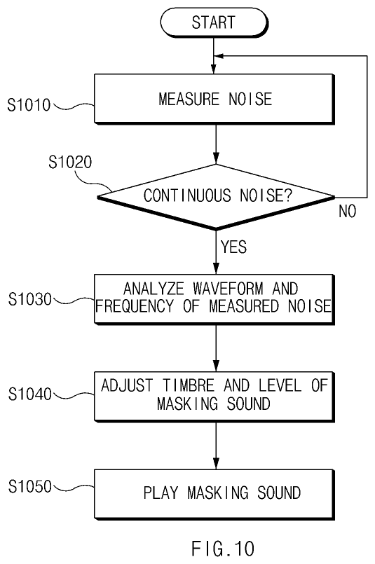

FIG. 10 is a flowchart illustrating a sound masking method according to embodiments of the present disclosure.

In operation S1010, a service platform server 100 of FIG. 1 may measure noise in real time using a sensor such as a microphone.

In operation S1020, the service platform server 100 may identify whether a type of the measured noise is continuous noise.

In operation S1030, the service platform server 100 may analyze a waveform and a frequency of the measured noise (YES in S1020). The service platform server 100 may analyze a maximum value, an average value, and a minimum value of the continuous noise.

In operation S1040, the service platform server 100 may adjust timbre and a level of a masking sound based on the result of analyzing the waveform and the frequency of the measured noise. The masking sound may be selected by a user or an AI technology.

In operation S1050, the service platform server 100 may play the adjusted masking sound. The service platform server 100 may output the played masking sound to the outside through the speaker.

Embodiments of the present disclosure may design and possess or transact a virtual sound in a virtual environment using a non-fungible token (NFT)-based blockchain technology.

Hereinabove, although the present disclosure has been described with reference to embodiments and the accompanying drawings, the present disclosure is not limited thereto. The present disclosure may be variously modified and altered by those having ordinary skill in the art to which the present disclosure pertains without departing from the spirit and scope of the present disclosure. Therefore, embodiments of the present disclosure are not intended to limit the technical spirit of the present disclosure but are provided only for the illustrative purpose. The scope of the present disclosure should be construed on the basis of the accompanying claims, and all the technical ideas within the scope equivalent to the claims should be included in the scope of the present disclosure.

What is claimed is:

1. A platform providing system for a vehicle content service, the platform providing system comprising:
a service platform server configured to construct a service platform in a metaverse-based virtual environment; and
a blockchain platform server configured to support the service platform,
wherein the service platform server is configured to:
generate a marketplace by means of a smart contract-based commerce platform in conjunction with the blockchain platform server,
generate a smart healing function based on a user experience analysis of a virtual vehicle in the service platform, and
generate a sound design platform supporting to design a virtual sound in the service platform and transact the virtual sound in the marketplace.

2. The platform providing system of claim 1, wherein the service platform server is configured to generate a transaction model supporting to perform a content transaction in conjunction with the blockchain platform server.

3. The platform providing system of claim 2, wherein the transaction model supports a content transaction using a non-fungible token (NFT)-based blockchain technology.

4. The platform providing system of claim 1, wherein the service platform server is configured to:
analyze a driving pattern of a user based on driving data of the virtual vehicle; and
establish an emotion modeling concept depending on the driving pattern.

5. The platform providing system of claim 4, wherein the service platform server is configured to:
generate determination logic for determining cyber sickness based on a biometric signal of the user using a biometric signal measurement device; and
generate cyber sickness mitigation control logic in response to that an occurrence of the cyber sickness.

6. The platform providing system of claim 5, wherein the biometric signal measurement device includes:
an electroencephalogram (EEG) measurement device configured to measure EEG in a dry manner; and
a haptic controller configured to measure a heart rate.

7. The platform providing system of claim 6, wherein the EEG measurement device includes:
a microphone configured to measure a breathing sound.

8. The platform providing system of claim 1, wherein the service platform server is configured to:
determine a user state by analyzing a biometric signal of a user;
derive an emotional concept based on the user state; and
generate sound masking emotion content using white noise or pink noise based on the emotional concept.

9. The platform providing system of claim 8, wherein the service platform server is configured to:
measure a sound level using a sensor installed on a front upper end in the interior of a vehicle;
play a masking sound based on the measured sound level; and
output the played masking sound through a front mid-range speaker of a vehicle.

10. The platform providing system of claim 8, wherein the service platform server is configured to:
measure a sound level using a sensor located at an ear position of the user;
play a masking sound based on the measured sound level; and
output the played masking sound through a speaker located at on the ear position of the user.

11. A platform providing method for a vehicle content service, the platform providing method comprising:
constructing, by a service platform server, a service platform in a metaverse-based virtual environment;
generating, by the service platform server, a marketplace by means of a smart contract-based commerce platform in conjunction with a blockchain platform server;
generating, by the service platform server, a smart healing function based on a user experience analysis of a virtual vehicle in the service platform; and
generating, by the service platform server, a sound design platform supporting to design a virtual sound in the service platform and transact the virtual sound in the marketplace.

12. The platform providing method of claim 11, wherein the constructing of the vehicle content platform includes:
generating a transaction model supporting to perform a content transaction in conjunction with the blockchain platform server.

13. The platform providing method of claim 12, wherein the transaction model supports a content transaction using a non-fungible token (NFT)-based blockchain technology.

14. The platform providing method of claim 11, wherein generating the smart healing function includes:
analyzing, by the service platform server, a driving pattern of a user based on driving data of the virtual vehicle; and
establishing, by the service platform server, an emotion modeling concept depending on the driving pattern.

15. The platform providing method of claim 14, wherein generating the smart healing function includes:
generating determination logic for determining cyber sickness based on a biometric signal of the user; and
generating cyber sickness mitigation control logic in response to that an occurrence of the cyber sickness.

16. The platform providing method of claim 11, wherein generating the sound design platform includes:
determine a user state by analyzing a biometric signal of a user;
deriving an emotional concept based on the user state; and
generating sound masking emotion content using white noise or pink noise based on the emotional concept.
